(19) United States
(12) Patent Application Publication
(10) Pub. No.: US 2016/0198725 A1
(43) Pub. Date: Jul. 14, 2016

(54) COMPOSITIONS AND METHODS FOR CONTROL OF MARINE ECTOPARASITES

(71) Applicant: SYLLETA INC., Toronto (CA)

(72) Inventors: Nikhil A. Gunari, Etobicoke (CA); Gilbert C. Walker, Mississuaga (CA)

(21) Appl. No.: 14/912,228

(22) PCT Filed: Aug. 15, 2014
(86) PCT No.: PCT/CA2014/000623

§ 371 Date: Feb. 16, 2016

Related U.S. Application Data
(60) Provisional application No. 61/866,244, filed on Aug. 15, 2013.

(51) Int. Cl. A01N 65/26; A61K 36/486; A23K 50/80; C09D 5/16; A01N 25/34; C09D 5/14; A61K 36/58; A01N 59/20

(52) U.S. Cl. CPC A01N 65/26; A61K 36/58; A61K 36/486; A01N 59/20; C09D 5/1618; A01N 25/34; C09D 5/14; A23K 50/80

(57) ABSTRACT

A limonoid composition for the prevention and control of marine ectoparasites on fish is provided herein. Said composition comprises at least one limonoid selected from the group consisting of azadirachtin, salannin, meliantriol, karanjin, and nimbin or is an extract from Neem, Milletia pinnata, or Azadirachta indica. Said composition may be deposited on a polymer which is in the form of a net or cage. Alternatively, said composition may be incorporated into a fish feed.

### RELATED APPLICATIONS

This application claims priority to U.S. Provisional Application No. 61/866,244, filed on Aug. 15, 2013, and which is incorporated herein by reference.

### FIELD OF THE INVENTION

The invention relates to the use of limonoids extracted from trees (leaf, bark, flower or fruit) in a formulation for removing or preventing the attachment of marine ectoparasites on to fish.

### BACKGROUND OF THE INVENTION

Marine Ectoparasites are organisms which inhabit the skin or outgrowths of the skin of another organism (the host) for various periods, and may be detrimental to the latter. Ectoparasites cause significant infestations in many kinds of fish. A wide variety of crustaceans feature prominently as fish ectoparasites and some species adversely affect fish stocks. Many of these ectoparasites (e.g. most lice) are host specific. Ectoparasitic Crustacea (Sea lice) constitute one of the most severe problems in the salmon and trout farming industry. Some common ectoparasites infecting fish are Invertebrata (Platyhelminthes, Trematoda), Annelida (Hirudinea) and Arthropoda (Crustacea, Copepoda).

In parasitic copepods, the body segments are often fused. The abdomen has four segments, which are usually all fused in the parasitic species as a result of adaptation to the host. These species are difficult to recognize as arthropods, let alone copepods. Approximately 8,000 species of copepods exist, most of which are free-living. These species are usually classified in seven orders. However, most parasitic copepods are found within three orders: Cyclopoida, Caligoida and Lernaeopodoida. All ectoparasitic copepods are known to feed on the blood and tissue fluids of the host. Sites of parasite attachment commonly become haemorrhagic, spongy and necrotic.

In a study conducted on parasitization of marine fish it was found that the mean prevalence of infection was 30.1%, the average of abundances was 6.7 parasites per fish. (Aspects of the Ecology of Metazoan Ectoparasites of Marine Fish, Int. Journal of Parasitology, 1995, 25(8), 945-978. Numerous (3947) individuals of 102 marine fish species from Papua New Guinea, New Zealand, the North Sea, Antarctica, the deep-sea and coast of southeastern Australia, Pacific Canada, Brazil, Argentina and the Great Barrier Reef were examined for metazoan ectoparasites).

The aquaculture industry is plagued with the marine ectoparasite problem. Sea lice is the common problem for the salmon and trout industry while the most frequent and abundant parasites for the sea-bass and sea-bream industry are monogeans Sparicotyle chrysophrii and Furnestinia echeneis, infecting the gills of bream, and Diplectanum aequans infecting the gills of bass. The copepod Caligus minimus can occasionally be found in the buccal cavity of bass.

Sea lice are naturally occurring marine ectoparasites that feed on the mucus, epidermal tissue, and blood of host marine fish. There are over 500 species and sea lice are found on most fish species worldwide. Sea lice infestations represent the most significant disease problem currently affecting sea-farmed salmon and trout around the world. These external parasites have been long recognized. Sea lice species such as Lepeophtheirus salmonis and various Caligus species have adapted to saline conditions and are major ectoparasites of farmed and wild Atlantic salmon and trout. FIG. 5 is a diagram illustrating sea lice life cycle.

Sea lice have a relatively simple life cycle with attached juveniles (parasitic) and mobile pre-adult (free-swimming) and adult stages on the host. Gravid females produce a series of egg strings, which give rise to three free-living planktonic stages before settlement on a host. The exact number of characteristic stages depends on the species. Lepeophtheirus salmonis has a total of ten stages, while Caligus elongatus, which does not have pre-adult stages, has eight. In both species, the copepodid is the infectious stage that locates and attaches to the host. Lepeophtheirus salmonis is characterized by a direct life cycle of five discrete phases and 10 stages that allows the approximate age of individual sea lice to be determined. The two main stages of this marine ectoparasite are (i) non-parasitic or free-swimming stage and (ii) parasitic or feeding stage. The ability of sea lice to find a host is limited by time and therefore distance from the potential host. The eggs hatch from gravid females into free-swimming nauplii. The interval between hatching (the naupliar stages) and infective capability (the parasitic/feeding stage) is approximately 4 days at 10° C. and 2 days at 15° C. The free-swimming larvae disperse via oceanic surface currents and then attach/infect fish to start the feeding stage.

Sea lice feed on fish skin, mucus and blood especially on the head, back and perianal region. Sea lice cause physical and enzymatic damage at their sites of attachment and feeding, untreated infestations may lead to death from severe erosion and exposure of subcutaneous tissues, secondary bacterial infections, osmotic imbalance and extreme stress. The effects of sea lice infestation can vary in their nature and severity depending upon a number of factors. These include infecting species, age and overall health of the fish. Parasitisation of fish can also cause an overall chronic stress response in fish since feeding and attachment cause changes in the mucus thickness and damage the epithelium resulting in loss of blood and fluids, and electrolyte changes. Parasitisation can decrease fish immune response and make them susceptible to other diseases and reduce growth and performance.

Managing sea lice is complicated because farmers must deal with a wide range of biological and environmental factors including the various life stages of lice and variances in water currents and temperatures. Some approved treatments don't work well on certain life stages of louse or in certain water temperatures. One approach to sea lice management is through an Integrated Pest Management Plan (IPMP). IPMP combines preventative farming practices with access to a variety of approved treatments that farmers can use strategically based on the life stage of the louse and environmental factors such as water temperatures. This approach allows farmers to use the right product at the right time. Sea lice treatments can be administered through feed, such as SLICE and Calicide or as a topical bath treatment. Bath treatments include Salmosan, hydrogen peroxide and AlphaMax. An environmentally friendly alternative used to control sea lice has focused on predation of sea lice by certain fish species. Natural predators of sea lice include leaner fish, which are used on fish farms in Norway, and to a smaller extent in Scotland. Their potential on a commercial scale to control sea lice has not been researched in depth in other fish farming nations.

There are potential disadvantages to using the above treatment methods. Bath treatments are more difficult and require more manpower to administer, requiring skirts and tarpaulins to be placed around the cages to contain the drug. This labor and time intensive process does not provide a high percentage of prevention of reinfestation especially to the significant size of operations. Recent use of well-boats containing the drugs has reduced both the concentration and environmental concerns, although transferring fish to the well boat and back to the cage can be stressful. Since the advent of SLICE (emamectin benzoate) bath treatments have not been used frequently. Emamectin benzoate is a Pyrethroid, which are direct stimulators of sodium channels in neuronal cells, inducing rapid depolarization and spastic paralysis leading to death in sea lice.

Even with all preventative measures and approved treatments, marine ectoparasite infestation is still a major problem to the aquaculture industry and causes colossal damage to the harvest. The evolved immunity towards the various commercial sea lice control compositions, compounds and formulations is a substantial challenge. The salmon and trout aquaculture industry in Chile, Norway, Canada and England are experiencing considerable financial losses. Thus, there is an immediate requirement in the aquaculture industry for novel methods to meet the challenge posed by the ever-growing immunity in sea lice populations towards the commercially available sea lice control methods.

### SUMMARY OF THE INVENTION

In an aspect, there is provided a composition for the control of marine ectoparasites comprising at least one limonoid

In a further aspect, there is provided fish feed comprising the composition described herein.

In a further aspect, there is provided a polymer soaked with the composition described herein.

In a further aspect, there is provided a method of controlling, inhibiting or preventing the attachment, propagation or growth of ectoparasites on fish comprising exposing the fish to the composition described herein.

In a further aspect, there is provided use of the composition described herein for controlling, inhibiting or preventing the attachment, propagation or growth of ectoparasites on fish.

### BRIEF DESCRIPTION OF FIGURES

These and other features of the preferred embodiments of the invention will become more apparent in the following detailed description in which reference is made to the appended drawings wherein:

FIG. 1 shows sea lice count at the farm site in the bay of Fundy after 60 days of Salmon being introduced into the water.

FIG. 2 shows sea lice count at the farm site in the bay of Fundy after 75 days of Salmon being introduced into the water.

FIG. 3 shows sea lice count at the farm site in the Bay of Fundy after 90 days of Salmon being introduced into the water.

FIG. 4 is sea lice count at the farm site in the bay of Fundy after 105 days of Salmon being introduced into the water.

FIG. 5 is a diagram illustrating sea lice life cycle.

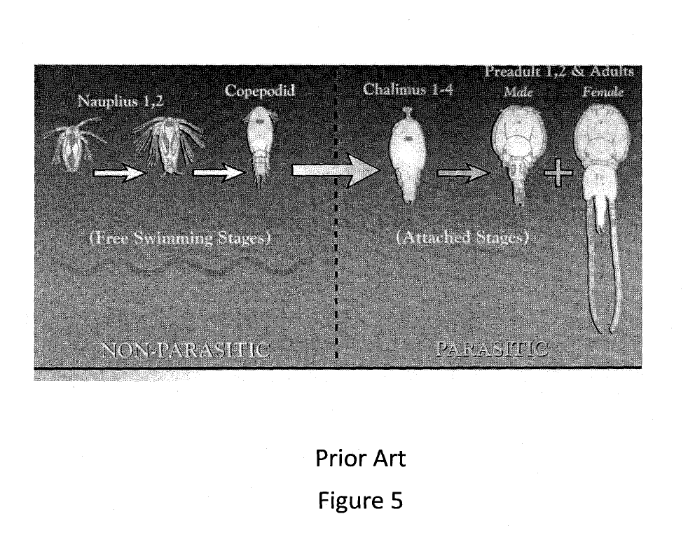

### DETAILED DESCRIPTION

In the following description, numerous specific details are set forth to provide a thorough understanding of the invention. However, it is understood that the invention may be practiced without these specific details.

The present invention provides the use of Limonoids and limnoid extracts (seed, leaf, bark, flower of trees) in the control of marine ectoparasite infestation in aqua culture facilities. More specifically, the invention concerns the use of one or more Limonoid extracts from the neem and Milletia pinnata trees for manufacturing a product for removing and/or preventing the attachment of marine ectoparasites on fish.

Limonoids are phytochemicals, abundant in citrus fruit and other plants of the families Rutaceae and Meliaceae. Chemically, the limonoids consist of variations of the furanolactone core structure. Limonoids are natural compunds generally found in skin of citrus fruits, seeds, leaves, flowers, bark of neem and karanja trees.

There is also provided methods of preventing or removing sea lice from fish, wherein a product comprising one or more neem tree extracts is administered.

In an aspect there is provided a composition for the control of marine ectoparasites comprising at least one limonoid.

In some embodiments, the limonoid is selected from the group consisting of azadirachtin, salannin, meliantriol, karanjin and nimbin, preferably azadirachtin and karanjin.

In some embodiments, the limonoid is extracted from a tree, preferably the seed, leaf, bark or flower of the plant. Preferably, the plant is neem or Milletia pinnata.

In some embodiments, the composition is Azadirachta indica extract.

In some embodiments, the composition is Milletia pinnata extract.

In some embodiments, the composition comprises the limonoid in a concentration from at least 1 ppm to 65,000 ppm.

In some embodiments, the composition further comprises at least one polymerized oil. Preferably, the at least one polymerized oil is selected from the group comprising a linseed oil , a perilla oil, a poppy seed oil, a soybean oil, a walnut oil, a tung oil, and mixtures thereof. In some embodiments, the composition comprises from >0 to 99% (v/v) of the at least one polymerized oil. In some embodiments, the at least one polymerized oil is a boiled oil having an iodine number greater than 120.

In some embodiments, the composition further comprises at least one essential oil derived from a fruit or a flower. Preferably, the at least one essential oil is selected from the group comprising limonene, lavender, rose and mixtures thereof.

In some embodiments, the composition further comprises linseed oil and limonene.

In some embodiments, the composition further comprises a block-copolymer capable of forming a micelle. Preferably, the block-copolymer is a biodegradable block copolymer, preferably polycaprolactone-block-poly(ethylene oxide)-block polycaprolactone triblock copolymer.

In some embodiments, the composition further comprises a foul release agent. Preferably, the foul release agent is polyvinylpyrrolidone [or Copper].

In an aspect there is provided a fish feed comprising the composition described herein.

In an aspect there is provided a polymer soaked with the composition described herein.

In some embodiments, the polymer is in the form of a net or cage.

In some embodiments, the at least one polymer is selected from the group consisting of a synthetic or a natural polymer.

In some embodiments, the at least one polymer is selected from the group consisting of nylon, high density polyethylene (HDPE), polyester, polyurethane, Teflon, cellulose and polypropylene.

In some embodiments, the composition is applied to the polymer by at least one of dipping, spraying, brushing, rolling and pouring the composition over the polymer.

In some embodiments, the composition is applied to the polymer during a cold drawing stage of producing a polymer fiber, preferably executed on a draw twister machine.

In an aspect there is provided a method of controlling, inhibiting or preventing the attachment, propagation or growth of ectoparasites on fish comprising exposing the fish to the composition described herein.

In some embodiments, the fish is exposed by feeding the fish the fish feed described herein.

In some embodiments, the fish is exposed by the addition of the composition to the fish's aquatic environment. Preferably, the fish's aquatic environment is a bath.

In some embodiments, the fish is exposed by housing the fish in the polymer described herein.

In some embodiments, the fish is a marine fish, preferably selected from Salmon, Trout and Bass.

In some embodiments, the ectoparasite is selected from Invertebrata (Platyhelminthes, Trematoda), Annelida (Hirudinea) and Arthropoda (Crustacea, Copepoda), preferably sea lice such as Lepeophtheirus Salmonis, Caligus minimus, monogeans Sparicotyle chrysophrii, Furnestinia echeneis or Diplectanum aequans.

In an aspect there is provided a use of the composition described herein for controlling, inhibiting or preventing the attachment, propagation or growth of ectoparasites on fish.

Applicant has observed that neem seed extracts, when applied to aquaculture cage nets made from polymer fibers, reduces the number of sea lice on salmon inside the cage.

Applicant has also observed that that neem seed extracts, when administered to sea lice in a beaker immobilized them in a short period of time.

Applicant has found that aquaculture cage nets treated with neem seed extracts not only prevented biofouling on nets but also resulted in substantially lower number of sea lice on salmon inside the cage as compared to the other cages treated with copper oxide. All cages were treated with the same dose of Ivermectin (sea lice control drug) throughout the test period, but only the neem seed extract treated nets had a significantly reduced number of sea lice on the salmon inside the cage. Aquaculture net cages/pens are most commonly made of polymer fibers. Most commonly used polymers are (but not limited to) nylon, polyester, polyethylene, high density polyethylene (Dyneema).

Applicant also found that direct administration of neem seed extracts in a beaker with seawater containing sea lice rendered them immobile within 15 minutes. Sea lice in control beakers that did not contain neem seed extract were active and swimming freely.

Neem seed extracts are chemically rich and have more than 300 compounds. Most of the active compounds are limonoids, found in the fruit, seeds, twigs, stem, and root bark.

So far, at least nine neem limonoids have demonstrated ability to deter feeding in insects, block insect growth, affecting a range of species that includes some of the most deadly pests of agriculture and human health. New limonoids are still being discovered in neem, but azadirachtin, salannin, meliantriol, and nimbin are the best known and, for now at least, seem to be the most significant.

Neem seed extracts have been used in the agriculture industry for decades as a biopesticide and has great potential in the fisheries industry. Recently neem seed extract has shown to be effective against bacteria especially Aeromonas hydrophila in goldfish, which causes ulcerative dermatitis. More recently Applicant has shown that neem tree extracts are very effective in controlling biofouling on aquaculture cage nets, as described in WO2012/139202, incorporated herein by reference.

Milletia pinnata is a species of tree in the pea family, Fabaceae, native in tropical and temperate Asia including parts of India, China, Japan, Malesia, Australia and Pacific islands. Karanjin, furanoflavanol, a type of limonoid, is obtained from the seeds of the karanja tree (Milletia pinnata).

Modes of action of Neem tree extracts against parasites can include: (i) feed deterrent, (ii) parasite growth regulation, (iii) reduction in longevity and fertility, (iv) reduced egg hatching mating disruption and (v) molting cycle disruption. Karanjin's primary mode of actions is feed deterrent and parasite growth regulation.

Generally speaking, the embodiments described herein are directed to a method of controlling marine ectoparasite infestation in the aquaculture industry, in particular controlling the attachment of sea lice in salmon and trout farming. As required, embodiments of the present invention are disclosed herein. However, the disclosed embodiments are merely exemplary, and it should be understood that the invention may be embodied in many various and alternative forms.

The advantages of the present invention are further illustrated by the following examples. The examples and their particular details set forth herein are presented for illustration only and should not be construed as a limitation on the claims of the present invention.

### EXAMPLES

100-meter smolt nylon nets were treated with neem seed extract (oil) and copper oxide based coating. Net treated with neem seed extracts reduced the number of sea lice on salmon inside the cage as shown in the table below. The data were collected over a period of 2 months.

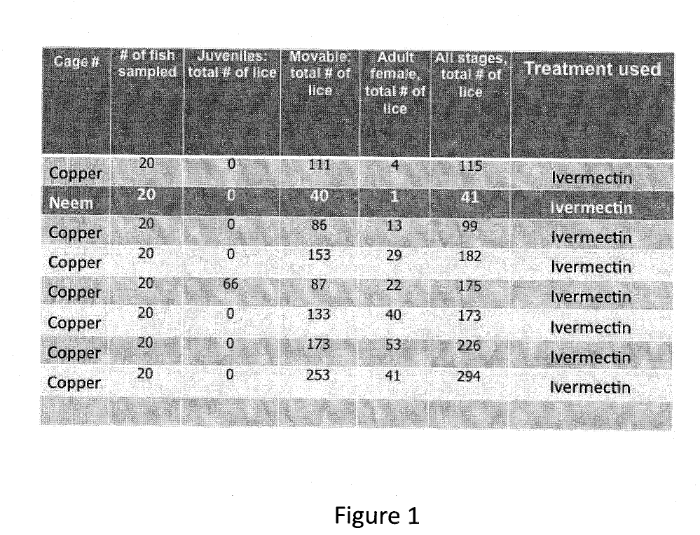

Referring to FIG. 1, there is shown sea lice count at the farm site in the Bay of Fundy after 60 days of Salmon being introduced into the water. Notably, with neem treatment (Row/Cage #2), the sea lice count is significantly reduced when compared with copper treatment.

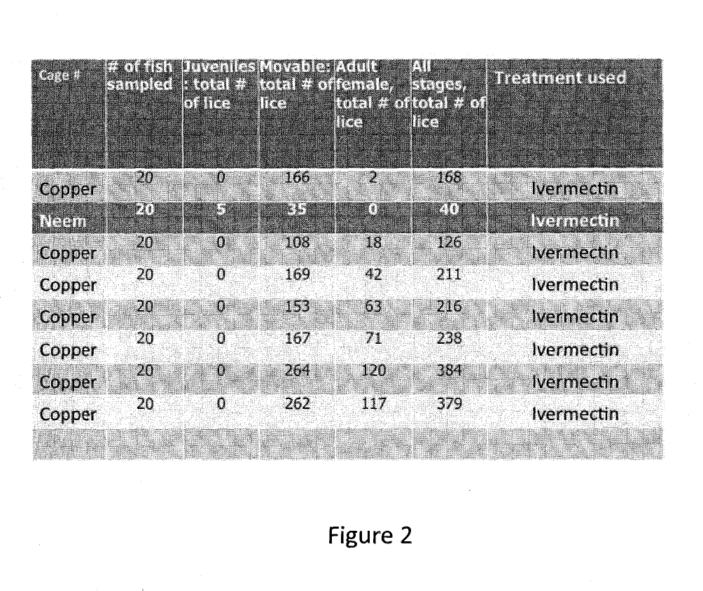

Referring to FIG. 2, there is shown sea lice count at the farm site in the Bay of Fundy after 75 days of Salmon being introduced into the water. Notably, with neem treatment (Row/Cage #2), the sea lice count is significantly reduced when compared with copper treatment.

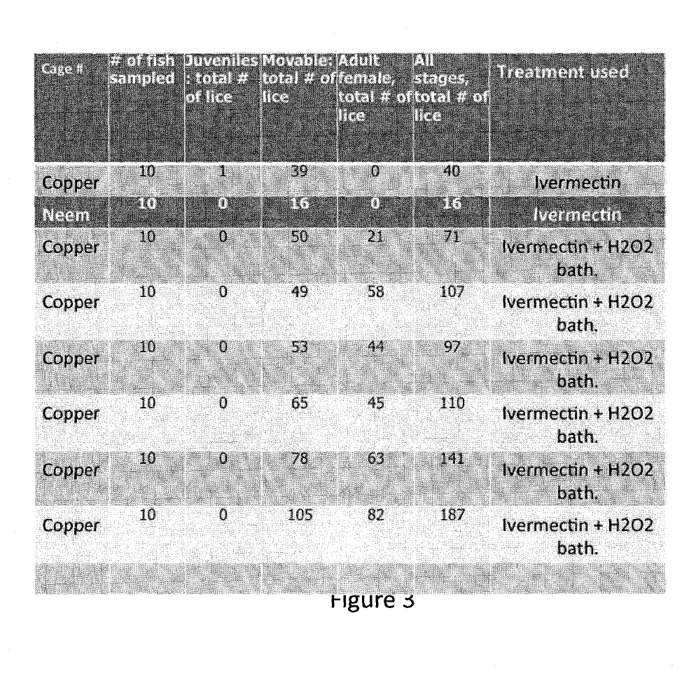

Referring to FIG. 3, there is shown sea lice count at the farm site in the Bay of Fundy after 90 days of Salmon being introduced into the water. Notably, with neem treatment (Row/Cage #2), the sea lice count is significantly reduced when compared with copper treatment (Row/Cage #1) and even with additional H₂O₂ treatment (Row/Cages #3-8).

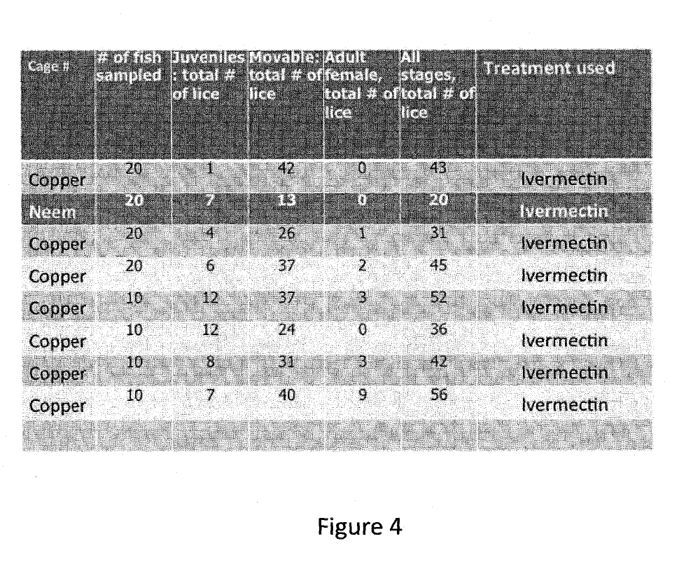

Referring to FIG. 4, there is shown sea lice count at the farm site in the Bay of Fundy after 105 days of Salmon being introduced into the water. Notably, with neem treatment (Row/Cage #2), the sea lice count is significantly reduced when compared with copper treatment.

The above demonstrates that formulations comprising a limonoid, e.g. neem oil, are effective for controlling, inhibiting or preventing the attachment, propagation or growth of ectoparasites on fish.

Although preferred embodiments of the invention have been described herein, it will be understood by those skilled in the art that variations may be made thereto without departing from the spirit of the invention or the scope of the appended claims. All documents disclosed herein are incorporated by reference.

1. A composition for the control of marine ectoparasites comprising at least one limonoid.

2. The composition of claim 1, wherein the limonoid is selected from the group consisting of azadirachtin, salannin, meliantriol, karanjin and nimbin, preferably azadirachtin and karanjin.

3. The composition of any one of claims 1 and 2, wherein the limonoid is extracted from a tree, preferably the seed, leaf, bark or flower of the plant.

4. The composition of claim 3, wherein the plant is neem or Milletia pinnata.

5. The composition of claim 1, wherein the composition is Azadirachta indica extract.

6. The composition of claim 1, wherein the composition is Milletia pinnata extract.

7. The composition of any one of claims 1 to 6, wherein the composition comprises the limonoid in a concentration from at least 1 ppm to 65,000 ppm.

8. The composition of claim 1 further comprising at least one polymerized oil.

9. The composition of claim 8, wherein the at least one polymerized oil is selected from the group comprising a linseed oil , a perilla oil, a poppy seed oil, a soybean oil, a walnut oil, a tung oil, and mixtures thereof.

10. The composition of any one of claims 8 and 9, wherein the composition comprises from >0 to 99% (v/v) of the at least one polymerized oil.

11. The composition of any one of claims 8-10, wherein the at least one polymerized oil is a boiled oil having an iodine number greater than 120.

12. The composition of any one of claims 1-11 further comprising at least one essential oil derived from a fruit or a flower.

13. The composition of claim 12, wherein the at least one essential oil is selected from the group comprising limonene, lavender, rose and mixtures thereof.

14. The composition of claim 13, further comprising linseed oil and limonene.

15. The composition of any one of claims 1-14, further comprising a block-copolymer capable of forming a micelle.

16. The composition of claim 15, wherein the block-copolymer is a biodegradable block copolymer, preferably polycaprolactone-block-poly(ethylene oxide)-block polycaprolactone triblock copolymer.

17. The composition of any one of claims 1-16, further comprising a foul release agent.

18. The composition of claim 17, wherein the foul release agent is polyvinylpyrrolidone [or Copper].

19. Fish feed comprising the composition of any one of claims 1-7.

20. A polymer soaked with the composition of any one of claims 1-18.

21. The polymer of claim 20, being in the form of a net or cage.

22. The polymer of claim 20 or 21, wherein the at least one polymer is selected from the group consisting of a synthetic or a natural polymer.

23. The polymer of claim 21, wherein the at least one polymer is selected from the group consisting of nylon, high density polyethylene (HDPE), polyester, polyurethane, Teflon, cellulose and polypropylene.

24. The polymer of any one of claims 21 to 23, wherein composition is applied to the polymer by at least one of dipping, spraying, brushing, rolling and pouring the composition over the polymer.

25. The polymer of any one of claims 21 to 24, wherein the composition is applied to the polymer during a cold drawing stage of producing a polymer fiber, preferably executed on a draw twister machine.

26. A method of controlling, inhibiting or preventing the attachment, propagation or growth of ectoparasites on fish comprising exposing the fish to the composition of any one of claims 1-18.

27. The method of claim 26, wherein fish is exposed by feeding the fish the fish feed of claim 19.

28. The method of claim 26, wherein the fish is exposed by the addition of the composition to the fish's aquatic environment.

29. The method of claim 28, wherein the fish's aquatic environment is a bath.

30. The method of claim 26, wherein the fish is exposed by housing the fish in the polymer of any one of claims 20-25.

31. The method of any one of claims 26-30, wherein the fish is a marine fish, preferably selected from Salmon, Trout and Bass.

32. The method of any one of claims 26-31, wherein the ectoparasite is selected from Invertebrata (Platyhelminthes, Trematoda), Annelida (Hirudinea) and Arthropoda (Crustacea, Copepoda), preferably sea lice such as Lepeophtheirus Salmonis, Caligus minimus, monogeans Sparicotyle chrysophrii, Furnestinia echeneis or Diplectanum aequans.

33. Use of the composition of any one of claims 1-18 for controlling, inhibiting or preventing the attachment, propagation or growth of ectoparasites on fish.
